(19) United States
(12) Patent Application Publication
(10) Pub. No.: US 2025/0213869 A1
(43) Pub. Date: Jul. 3, 2025

(54) METHOD AND APPARATUS FOR DETECTING ATRIAL TACHYCARDIA, ELECRONIC DEVICE AND STORAGE MEDIUM

(71) Applicant: CORERHYTHM MEDICAL TECHNOLOGY (HANGZHOU) CO., LTD, Hangzhou, Zhejiang (CN)

(72) Inventors: Min HUANG, Hangzhou (CN); Jianqu XIONG, Hangzhou (CN); Guiling LI, Hangzhou (CN); Jianfei WANG, Hangzhou (CN); Cheng FANG, Hangzhou (CN); Lian JIN, Hangzhou (CN); Zhen ZHOU, Hangzhou (CN)

(73) Assignee: CORERHYTHM MEDICAL TECHNOLOGY (HANGZHOU) CO., LTD, Hangzhou, Zhejiang (CN)

(21) Appl. No.: 18/852,783

(22) PCT Filed: Apr. 25, 2023
(86) PCT No.: PCT/CN2023/090553

§ 371 Date: Sep. 30, 2024

(30) Foreign Application Priority Data
May 19, 2022 (CN) 202210557762.6

(51) Int. Cl. A61N 1/362; A61N 1/365

(52) U.S. Cl. CPC A61N 1/3624; A61N 1/365

(57) ABSTRACT

A method for detecting atrial tachycardia includes monitoring a total number of historical detection intervals belonging to atrial tachycardia intervals within a set time of a target pacemaker in a tracking mode. If the total number is within a first preset number range threshold an atrioventricular delay interval duration of the target pacemaker is delayed. The method further includes determining whether the total number of the historical detection intervals belonging to the atrial tachycardia intervals within the set time after the atrioventricular delay interval duration of the target pacemaker is adjusted is greater than an upper limit value of the first preset number range threshold. If the total monitored number of the historical detection intervals is greater than the upper limit value of the first preset number range threshold a determination is made that an atrial tachycardia event has occurred.

### CROSS-REFERENCE TO RELATED APPLICATIONS

The present application claims priority to Chinese Patent Application No. 202210557762.6, filed on May 19, 2022, the entire contents of which are hereby incorporated into the present application by reference.

### TECHNICAL FIELD

The present application relate to that field of clinical medicine, in particular to a method and an apparatus for detecting atrial tachycardia.

### TECHNICAL BACKGROUND

Implantable medical equipment is an electronic therapeutic instrument implantable in the body, which emits electric pulses powered by batteries through a pulse generator, and stimulates the myocardium contacted by the electrode through conduction of a wire electrode, and causes the heart to be excited and contracted, thus achieving the purpose of treating cardiac dysfunction caused by certain arrhythmias. In the dual-chamber pacing mode of atrial tracking, in the case that the atrial rate is high enough to a certain extent, the pacemaker will work in a 2:1 conduction mode to cause rapid ventricular pacing, and in the case that the ventricular pacing of the pacemaker works at this rate for a long time, it will cause discomfort to patients. In order to solve this phenomenon, the current common practice is to design a mode switching function. in the case that the pacemaker detects atrial tachycardia, it will control the pacemaker to switch from atrial tracking mode to atrial non-tracking mode. In atrial non-tracking mode, the ventricular pacing rate will remain at or slightly higher than the base rate, and keep this mode until the atrial tachycardia stops, and then the pacemaker switches back to atrial tracking mode. At present, the existing detection schemes for atrial tachycardia include: 1. detect atrial rate, and consider whether atrial tachycardia occurs in the case that the atrial rate exceeds the preset value; 2. combined with the analysis of atrial and ventricular association, atrial tachycardia is suspected when N:1 association occurs, but both detection methods have blind areas in the detection of post ventricular atrial blank (PVAB), and there may be cases where atrial tachycardia events are not detected, which will affect the accuracy of detection.

### SUMMARY OF THE INVENTION

Therefore, the technical problem to be solved in the present application is to overcome the defects that the detection and analysis methods in the prior art have blind areas, and atrial tachycardia hidden in the post ventricular atrial blank can not be effectively found in the case that detecting atrial tachycardia events, which leads to the long-term high working rate of implantable medical equipment, thus causing discomfort to patients, thus providing a method and a device for detecting atrial tachycardia.

According to a first aspect, an embodiment of the present application provides a method for detecting atrial tachycardia, which comprises the following steps:
monitoring the total number of atrial tachycardia intervals in each historical detection interval of the target pacemaker within the set time in the tracking mode;
in the case that the total number is within the threshold of a first preset number range, adjusting the atrial-ventricular delay interval duration of the target pacemaker;
determining whether the total number of historical detection intervals belonging to atrial tachycardia intervals within the set time after the atrial-ventricular delay interval duration of the target pacemaker is adjusted is greater than the upper limit value of a first preset number range threshold;
in the case that the total number of historical detection intervals belonging to atrial tachycardia intervals within the set time after the atrial-ventricular delay interval of the target pacemaker is adjusted is greater than the upper limit value of the first preset number range threshold, it is determined that atrial tachycardia events occur.

Optionally, before determining whether the total number of historical detection intervals belonging to atrial tachycardia intervals within the set time after the atrial-ventricular delay interval duration of the target pacemaker is adjusted is greater than the upper limit value of the first preset number range threshold, the method further comprises:
starting the confirmation cycle of atrial tachycardia;
acquiring the detection sequence of the current detection interval, and determining whether the detection sequence is consistent with the target sequence;
in the case that the detection sequence is consistent with the target sequence, maintaining the adjustment of the atrial-ventricular delay interval duration of the target pacemaker;
determining whether the current detection interval is the atrial tachycardia interval;
in the case that the current detection interval is the atrial tachycardia interval and the current detection interval is within the atrial tachycardia confirmation period, it is judged whether the total number of historical detection intervals belonging to atrial tachycardia intervals within the set time after the atrial-ventricular delay interval duration of the target pacemaker is adjusted is greater than the upper limit value of the threshold of a first preset number range.

Optionally, the monitoring of the total number of atrial tachycardia intervals in each historical detection interval in the set time of the target pacemaker in the tracking mode includes:
acquiring atrial sensing events in the current historical detection interval within a set time;
determining whether the rate of the atrial sensing event exceeds the detect rate of atrial tachycardia;
determining that the current historical detection interval is an atrial tachycardia interval in the case that the rate of the atrial sensing event exceeds the atrial tachycardia detect rate;
counting the total number of all atrial tachycardia intervals.

Optionally, before counting the total number of all atrial tachycardia intervals, the method further comprises:
in the case that the atrial sensing event rate does not exceed the atrial tachycardia detect rate, determining whether the atrial sensing event rate is greater than a second detect rate, and the second detect rate is less than the atrial tachycardia detect rate;
in the case that that rate of the atrial sensing event is greater than the second detect rate, determine that the current historical detection interval corresponding to the atrial sensing event is a preset number of atrial tachycardia interval, wherein the preset number is greater than 0 and less than 1.

Optionally, adjusting the atrial-ventricular delay interval duration of the target pacemaker comprises:
obtaining the durations of the post ventricular atrial blank period and the atrioventricular interval;
adjusting the atrial-ventricular delay interval duration based on the relationship between the current detection interval and the durations of the post ventricular atrial blank period and atrioventricular interval.

Optionally, in the case that the total number is greater than the upper threshold value of the first preset number range, the method further comprises:
determining whether the number of atrial tachycardia intervals identified as integers in the total number is greater than a second preset number threshold;
in the case that that number is greater than the second preset number threshold, determining whether a ventricular event occurs in each historical detection interval in which the atrial tachycardia interval is identify as an integer within the set time of the monitoring target pacemaker in the tracking mode;
in the case that the number of ventricular events not occurring in the historical detection interval is greater than the second preset number threshold, it is determined that atrial tachycardia events occur.

Optionally, the method further comprises:
adjusting the target pacemaker from a tracking mode to a non-tracking mode after determining that an atrial tachycardia event occurs.

According to a third aspect, an embodiment of the application provides an electronic device, comprising:
a memory and a processor, wherein the memory and the processor are in communication connection with each other, and computer instructions are stored in the memory, and the processor executes the method described in the first aspect or any optional embodiments of the first aspect by executing the computer instructions.

According to a fourth aspect, an embodiment of the application provides a computer-readable storage medium, wherein computer instructions are stored, and the computer instructions are used for causing the computer to execute the method described in the first aspect or any optional embodiments of the first aspect.

The technical scheme of the application has the follow advantages:
according to the method and the apparatus for detecting atrial tachycardia provided by the application, the total number of historical detection intervals belonging to atrial tachycardia intervals in the set time of the target pacemaker in the tracking mode is monitored; in the case that the total number is within the threshold of a first preset number range, adjusting the atrial-ventricular delay interval duration of the target pacemaker; determining whether the total number of historical detection intervals belonging to atrial tachycardia intervals within the set time after the atrial-ventricular delay interval duration of the target pacemaker is adjusted is greater than the upper limit value of a first preset number range threshold; in the case that the total number of historical detection intervals belonging to atrial tachycardia intervals within the set time after the atrial-ventricular delay interval duration of the target pacemaker is adjusted is greater than the upper limit value of the first preset number range threshold, it is determined that atrial tachycardia event occurs. By detecting the total number of historical detection intervals belonging to atrial tachycardia intervals within a set time, in the case that the total number is at the first preset number range threshold, it is suspected that atrial tachycardia event has occurred, and the detection interval hidden in the post ventricular atrial blank period is detected by adjusting the atrial-ventricular delay interval duration of the target pacemaker. in the case that the current detection interval is detected as atrial tachycardia interval, continue to judge whether the total number of historical detection intervals belonging to atrial tachycardia intervals in the set time after the atrial-ventricular delay interval duration of the target pacemaker is adjusted is greater than the upper limit value of the first preset number range threshold, and in the case that the total number of historical detection intervals belonging to atrial tachycardia intervals in the set time after the atrial-ventricular delay interval duration of the target pacemaker is adjusted is greater than the upper limit value of the first preset number range threshold, it can be determined that atrial tachycardia events have occurred. By adjusting the atrial-ventricular delay interval duration of the target pacemaker, hidden atrial tachycardia events can be effectively detected, which improves the accuracy of atrial tachycardia event detection, thus greatly reducing the occurrence of discomfort caused by the long-term high working rate of the target pacemaker.

### BRIEF DESCRIPTION OF APPENDED DRAWINGS

In order to explain the specific embodiments of the present application or the technical solutions in the prior art more clearly, the appended drawings needed in the description of the specific embodiments or the prior art will be briefly introduced below. Apparently, the appended drawings in the following description are some embodiments of the present application. For the person skilled in this field, other drawings can be obtained according to these appended drawings without expenditure of creative labor.

FIG. 1 is a flowchart of a method for detecting atrial tachycardia according to an embodiment of the present application;

FIG. 2 is a system block diagram of an implantable medical device for detecting atrial tachycardia according to an embodiment of the present application;

FIG. 3 is a working flow chart of the atrial tachycardia protection window of the method for detecting atrial tachycardia according to the embodiment of the present application;

FIG. 4 is a schematic structural diagram of an apparatus for detecting atrial tachycardia according to an embodiment of the present application;

FIG. 5 is a schematic structural diagram of an electronic device according to an embodiment of the present application.

### DETAILED DESCRIPTION

The technical scheme of the present application will be described clearly and completely with the appended drawings. Apparently, the described embodiments are a part of the embodiments of the present application, not all of them. Based on the embodiments in the present application, all other embodiments obtained by persons skilled in this field without expenditure of creative labor belong to the protection scope of the present application.

In the description of the present application, it should be noted that the orientation or positional relationship indicated by the terms “center”, “upper”, “lower”, “left”, “right”, “vertical”, “horizontal”, “inner” and “outer” are based on the orientation or positional relationship shown in the appended drawings, and are only for the purpose of facilitating the description of the present application and simplifying the description, rather than indicating or implying that the device or element referred to must have a particular orientation, be constructed and operated in a particular orientation, it is not to be construed as a limitation of the present application. In addition, the terms “first” and “second” are only used for descriptive purposes and cannot be construed as indicating or implying relative importance.

In the description of the present application, it should be noted that unless otherwise specified and limited, the terms “installation”, “link” and “connection” should be broadly understood, for example, they can be fixed connection, detachable connection or integrated connection; It can be a mechanical connection or an electrical connection; It can be directly connected, indirectly connected through an intermediate medium, or internally connected between two elements, wirelessly connection or wired connection. For persons skilled in the art, the specific meanings of the above terms in the present application can be understood in specific circumstances.

In addition, the technical features involved in different embodiments of the present application described below can be combined with each other as long as they do not conflict with each other.

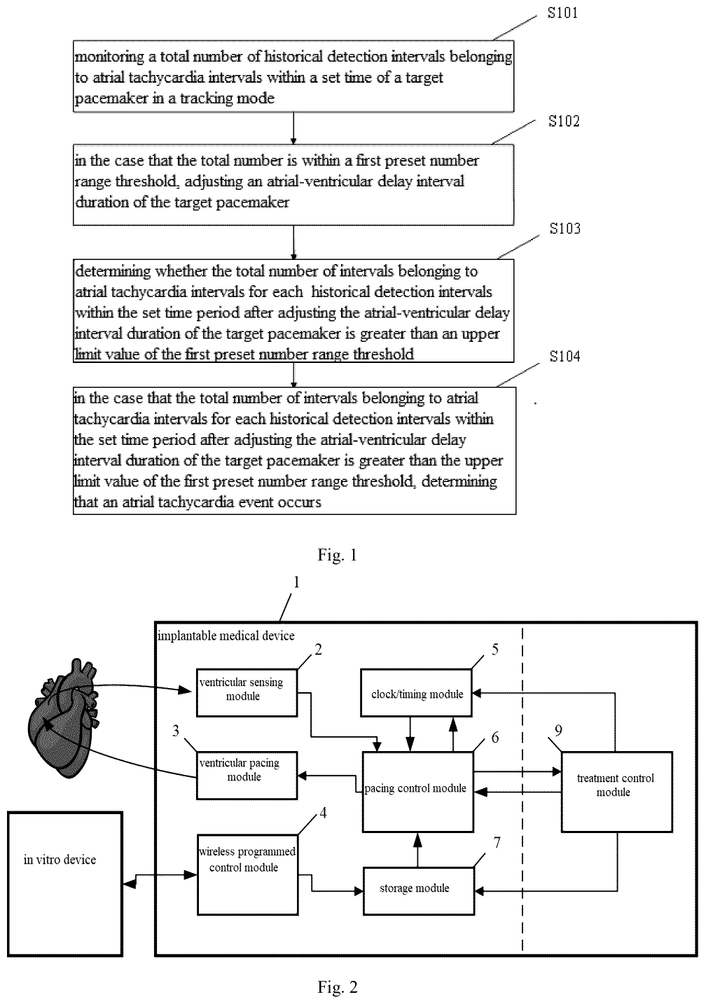

The embodiments of the present application provide a method for detecting atrial tachycardia, as shown in FIG. 1, which specifically comprise the following steps:

Step S101: monitoring a total number of historical detection intervals belonging to atrial tachycardia intervals within a set time of a target pacemaker in a tracking mode.

Specifically, in practical application, the target pacemaker always works in a tracking mode, and opens an Atrial Tachycardia Protection Window (ATPW) in the case that the target pacemaker detects a first atrial rate which exceeds the Atrial Tachycardia Detect Rate (ATDR), and the protection window is opened after each subsequent atrial sensing event until N consecutive atrial rates are lower than the atrial tachycardia detect rate. Optionally, the value of N can be 8, but the embodiment of the present application is not limited to this. By opening the protection window, the atrial pacing pulse issuing can be suppressed in the case of atrial tachycardia, and the ventricular pacing rate will also work according to the basic rate or the sensor indication rate. The length of the protection window is M % of the interval length corresponding to the current atrial tachycardia detect rate. Illustratively, the value of M can be 70. The problem that atrial pacing events issued by pacemakers may lead to misjudgment of atrial tachycardia events in the case that atrial tachycardia is suspected can be solved.

Step S102: in the case that the total number is within a first preset number range threshold, adjusting an atrial-ventricular delay interval duration of the target pacemaker.

In practical application, illustratively, the first preset number range threshold in the embodiment of the present application is 3-5 in the last 8 historical detection intervals. In the case that the total number is within the first preset number range threshold and there is an “atrial tachycardia suspecting window” (for example, 8 seconds) continuously, it is suspected that an atrial tachycardia event may occur. However, due to the presence of shielding of post ventricular atrial blanking period, the actual situation cannot be detected. The embodiment of the present application continues to detect atrial sensing events by adjusting the Atrial-Ventricular Delay (AVD) duration of the target pacemaker, which solves the problem that the existence of post ventricular atrial blanking period may lead to misjudgment of atrial tachycardia events when atrial tachycardia is suspected.

Specifically, in practical application, in the case that the total number is within the first preset number range threshold and the “atrial tachycardia suspecting window” is continuous, atrial tachycardia event is suspected to occur. By adjusting the atrial-ventricular delay interval duration, atrial sensing events in the post ventricular atrial blanking periods can be detected, and the judgment accuracy of atrial tachycardia events can be improved.

Step S103: determining whether the total number of intervals belonging to atrial tachycardia intervals for each historical detection intervals within the set time period after adjusting the atrial-ventricular delay interval duration of the target pacemaker is greater than an upper limit value of the first preset number range threshold.

Specifically, in practical application, after the atrial-ventricular delay interval duration of the target pacemaker is adjusted, the total number of intervals belonging to atrial tachycardia intervals for each historical detection intervals is counted to determine whether the total number of atrial tachycardia intervals is greater than the upper limit value of the first preset number range threshold, illustratively, the upper limit value is 5.

Step S104: in the case that the total number of intervals belonging to atrial tachycardia intervals for each historical detection intervals within the set time period after adjusting the atrial-ventricular delay interval duration of the target pacemaker is greater than the upper limit value of the first preset number range threshold, determining that an atrial tachycardia event occurs.

Specifically, in practical application, after the atrial-ventricular delay interval duration of the target pacemaker is adjusted, atrial sensing events in the post ventricular atrial blanking period will be detected, and atrial tachycardia interval may exist in the historical detection interval in the post ventricular atrial blanking period. By counting again the total number of intervals belonging to atrial tachycardia intervals for historical detection intervals after adjusting the atrial-ventricular delay interval duration, in the case that the total number is greater than the upper limit value of the first preset number range, it is determined that the detection criterion 1 is satisfied, and atrial tachycardia can be determined to have occurred.

In the embodiment of the application, when the upper limit value of the first preset number range is 5, it will be explained as an example. After adjusting the atrial-ventricular delay duration of the target pacemaker, each historical detection interval in the set time period is determined, in the case that the total number of atrial tachycardia intervals in the latest 8 historical detection intervals is 5, it is determined that atrial tachycardia has occurred, but the actual situation is not limited to this. The upper limit value of the first preset quantity can be set according to the actual situation.

By performing the above steps, the method for detecting atrial tachycardia provided by the embodiment of the present application, by monitoring a total number of historical detection intervals belonging to atrial tachycardia intervals within a set time of a target pacemaker in a tracking mode; in the case that the total number is within a first preset number range threshold, adjusting an atrial-ventricular delay interval duration of the target pacemaker; determining whether the total number of intervals belonging to atrial tachycardia intervals for each historical detection intervals within the set time period after adjusting the atrial-ventricular delay interval duration of the target pacemaker is greater than an upper limit value of the first preset number range threshold; in the case that the total number of intervals belonging to atrial tachycardia intervals for each historical detection intervals within the set time period after adjusting the atrial-ventricular delay interval duration of the target pacemaker is greater than the upper limit value of the first preset number range threshold, an atrial tachycardia event to occur is determined. The detection interval in the post ventricular atrial blanking period is detected by adjusting the atrial-ventricular delay duration of the target pacemaker; in the case that the total number of intervals belonging to atrial tachycardia intervals for each historical detection intervals within the set time period is greater than the upper limit value of the first preset number range threshold, it can be determined that atrial tachycardia event has occurred. By adjusting the atrial-ventricular delay of the target pacemaker, the hidden atrial tachycardia event can be effectively detected, which not only improves the detection accuracy of the atrial tachycardia event, but also greatly reduces the occurrence of the patient's discomfort caused by the target pacemaker being at the high operating rate for a long time.

Specifically, in one embodiment, the above step S101 of monitoring a total number of historical detection intervals belonging to atrial tachycardia intervals within a set time of a target pacemaker in a tracking mode, specifically comprises the following steps:

Step S201: acquiring an atrial sensing event in the current historical detection interval within a set time period.

Specifically, in practical application, after the first atrial rate that exceeds the atrial tachycardia detect rate (ATDR) is detected, the “atrial tachycardia protection window” will be opened, and each subsequent detection interval will be monitored to obtain atrial sensing event in each detection interval. The “atrial tachycardia protection window” provided by the embodiments of the present application can effectively avoid the possible maintaining or aggravating of atrial tachycardia events and the interference of atrial tachycardia events detection by atrial pacing events.

Step S202: determining whether a rate of the atrial sensing event exceeds an atrial tachycardia detect rate.

Specifically, in practical application, the atrial tachycardia detect rate can be dynamically adjusted according to the current basic rate or the sensor indication rate (when the sensor indication rate function is turned on), and Xppm is added on the basis of the current basic rate or the sensor indication rate, wherein Xppm is a doctor's programmed value and can be set within a certain range (such as 0-1000 ppm). Illustratively, X can be taken as 35, 40, 45, etc., and the rate is not lower than the upper limit of tracking rate.

Specifically, in practical application, the basic rate can be programmed and set by doctors according to different conditions of patients. For example, the basic rate can be 60 ppm, but the numerical value of the basic rate in the embodiment of the present application is not limited to this.

When the patient is in a state of exercise, such as fast walking and running, the atrial tachycardia detect rate can be dynamically adjusted according to the different conditions of the patient, which solves the problem that the atrial tachycardia rate can not correctly evaluate whether the patient has atrial tachycardia events during exercise.

Step S203: determining that the current historical detection interval is an atrial tachycardia interval in the case that the rate of the atrial sensing event exceeds the atrial tachycardia detect rate.

Specifically, in practical application, an interval is inversely proportional to rate, that is, in the case that the rate of the atrial sensing event exceeds the atrial tachycardia detect rate, the current historical detection interval (atrial interval, that is, AA interval) will be lower than that of atrial tachycardia detect rate, that is, the current AA_intvl is lower than that of “atrial tachycardia detect rate”, then this AA_intvl is defined as an “atrial tachycardia interval”.

Step S204: counting a total number of all atrial tachycardia intervals. Specifically, in practical application, the latest eight AA intervals are determined, and the total number of atrial tachycardia intervals in these eight AA intervals is calculated according to the above detection criterion 1. In the case that the total number is greater than the upper limit value of the first preset number range, it is determined that the detection criterion 1 is satisfied, and it can also be determined that an atrial tachycardia event has occurred.

Specifically, in an embodiment, before executing the step of S204, the method for detecting atrial tachycardia provided by the embodiment of the present application specifically further comprises the following steps:

Step S301: in the case that the rate of the atrial sensing event does not exceed the atrial tachycardia detect rate, determining whether the rate of the atrial sensing event is greater than a second detect rate, wherein the second detect rate is less than the atrial tachycardia detect rate.

Specifically, in practical application, the existing determination of atrial tachycardia events does not take into account that the current historical detection interval is lower than the atrial tachycardia detection interval, there may also be atrial tachycardia events, that is, there is no comprehensive consideration of the possible occurrence of atrial tachycardia. Based on the detection criterion of the prior art, the embodiments of the present application fully consider the gaps in the existing detection criterion, improve the detection criterion 1, and greatly improve the detection accuracy.

Step S302: in the case that the rate of the atrial sensing event is greater than the second detect rate, determining that the current historical detection interval corresponding to the atrial sensing event is a preset number of atrial tachycardia intervals, wherein the preset number is greater than 0 and less than 1.

Specifically, in practical application, in order to improve the detection accuracy, the embodiments of the present application supplement the detection criterion 1, as follows:
(1) If the rate corresponding to the current AA interval exceeds the “atrial tachycardia detect rate”, then the AA_intvl is defined as an “atrial tachycardia interval”;
(2) If in the current AA interval, (AA_intvl/2) is less than the AA interval (ATDR_intvl) of the “atrial tachycardia detect rate”, and (AA _ intvl/2) is less than (SAV+PAVB), then the AA_intvl is defined as 0.5 “atrial tachycardia intervals”, wherein SAV is Sended AV Interval, PAVB is Post Atrial Ventricular Blanking.

By perfecting and supplementing the detection criterion 1, the embodiments of the present application solve the problem that it can not accurately evaluate atrial tachycardia events by judging atrial rate alone.

Specifically, in an embodiment, in the case that the total number is greater than the upper limit value of the first preset number range, it further comprises the following steps:

Step S401: determining whether a number of atrial tachycardia intervals the value of which is identified as integer in the total number is greater than a second preset number threshold.

Step S402: in the case that the number is greater than the second preset number threshold, determining whether a ventricular event occurs in each historical detection interval in which the value of the atrial tachycardia interval is identified as an integer within the set time period of the monitoring target pacemaker in the tracking mode.

Step S403: in the case that a number of ventricular events not occurring in the historical detection intervals is greater than the second preset number threshold, determining that an atrial tachycardia event occurs.

Specifically, in practical application, the total number is statistically determined by (1) and (2) in detection criterion 1. In the case that the total number is greater than the upper limit value 5 of the first preset number threshold, the number of atrial tachycardia intervals identified as integers in the total number will be calculated to determine whether it is greater than the second preset number range. In the case that the number is greater than the second preset number range, determine whether a ventricular event occurs in each historical detection interval identified as an integer, and determine that an atrial tachycardia event occurs when the number of ventricular events not occurring in the historical detection interval is greater than the second preset number threshold.

Illustratively the second preset number threshold can be 3, that is, in the case that the number of atrial tachycardia intervals identified as integers is 3, the atrial tachycardia intervals identified as integers are determined to determine whether issuing ventricular events occur, and in the case that the number of said ventricular events not occurring is greater than 3, it is determined that atrial tachycardia events occur.

Specifically, in practical application, in order to further improve the detection accuracy, the embodiments of the present application add the content of detection criterion 2 on the basis of detection criterion 1, and combine detection criterion 1 and detection criterion 2 to detect and determine whether atrial tachycardia events occur, that is, in the case that the target pacemaker meets detection criterion 1 and detection criterion 2 at the same time, it is considered that atrial tachycardia events are detected. Through double determination, the detection accuracy is further improved.

Specifically, the content of detection criterion 2 is as follows:
determining the latest eight AA intervals, in the case that there is an AA interval exceeding the second preset number range, it is an atrial tachycardia interval and there is no ventricular event in the AA interval. Optionally, the second preset quantity value involved in detection criterion 2 may be 3, that is, in the case that three or more AA intervals out of the latest eight AA intervals are atrial tachycardia intervals and there is no conduction ventricular event, it is determined that detection criterion 2 is satisfied.

Specifically, in practical application, when determining detection criterion 2, the condition of determining whether AA interval is atrial tachycardia interval refers to (1) in detection criterion 1, that is, AA interval in (2) of detection criterion 1 does not participate in the determination of detection criterion 2 because it is a non-integer AA interval.

When the target pacemaker detects the latest eight AA intervals, if the detection criterion 1 and 2 are satisfied at the same time, it is determined that the atrial tachycardia event is detected; in the case that the detection criterion 1 and 2 are not satisfied at the same time, it is determined that atrial tachycardia has not been detected, and the target pacemaker will continue to work in tracking mode.

Specifically, in an embodiment, when the above step S102 is executed, adjusting the atrial-ventricular delay duration of the target pacemaker specifically further comprises the following steps:

Step S501: obtaining durations of an post ventricular atrial blanking period and an atrioventricular interval.

Step S502: adjusting the atrial-ventricular delay duration based on a relationship between the current detection interval and the durations of the post ventricular atrial blanking period and the atrioventricular interval.

In the case that one of the two criteria is satisfied and persists in the “atrial tachycardia suspicion window” period, it is suspected that there is shielding of post ventricular atrial blanking period, which leads to the situation that atrial tachycardia events occur but can not be detected. At this time, the atrial-ventricular delay of the target pacemaker will be adjusted to detect the shielding period.

Specifically, the set time of the continuous “atrial tachycardia suspicion window” can be 8s, but the actual situation is not limited to this.

Specifically, in practical application, the embodiments of the present application provide two adjustment strategies, so as to better detect the shielding period and avoid the situation to occur that there exists a blind area in the detection and the atrial tachycardia event in the blind area cannot be detected due to the presence of shielding of post ventricular atrial blanking period, which ultimately affects the accuracy of detection.

Specifically, in practical application, the specific contents of the adjustment strategy are as follows:

Adjustment strategy 1: If the current minimum AA interval is greater than SAVD+(PVAB/2) and less than SAVD+PVAB, decrease AVD=AVD−(PVAB/2), wherein the minimum AVD is not less than 30 ms.

Adjustment strategy 2: If the current minimum AA interval is greater than SAVD and less than SAVD+½PVAB, expand AVD=AVD+(PVAB/2), and the maximum AVD is not more than 300 ms.

Wherein, SAVD is an atrioventricular interval; PVAB is a post ventricular atrial blanking period; wherein AVD comprises SAVD and PAVD (Pacing atrial-ventricular delay).

Specifically, in an embodiment, before the above step S103 of determining whether the total number of intervals belonging to atrial tachycardia intervals for each historical detection intervals within the set time period after adjusting the atrial-ventricular delay interval duration of the target pacemaker is greater than an upper limit value of the first preset number range threshold, it further specifically comprises the following steps:

Step S601: starting a confirmation period of atrial tachycardia.

Specifically, in practical application, in order to ensure the effectiveness of detection, the embodiment of the present application sets a confirmation period of atrial tachycardia. In the case that the total number is within the first preset number range threshold, it is suspected that there is shielding of the post ventricular atrial blanking period, and then the confirmation period of atrial tachycardia will be started, and the period length can be programmed controlled by the doctor. Illustratively, the period length can be 10s, but the embodiment of the present application is not limited to this.

Step S602: acquiring a detection sequence of a current detection interval, and determining whether a sequence of the detection sequence is consistent with that of a target sequence.

Illustratively, in practical application, the target sequence for adjusting strategy 1 can be AS/AP-VP-AR.

Step S603: in the case that the sequence of the detection sequence is consistent with that of the target sequence, maintaining the adjustment of the atrial-ventricular delay duration of the target pacemaker.

Step S604: determining whether the current detection interval is the atrial tachycardia interval.

Step S605: in the case that the current detection interval is the atrial tachycardia interval and the current detection interval is within the confirmation period of atrial tachycardia, determining whether the total number of intervals belonging to atrial tachycardia intervals for each historical detection intervals within the set time period after adjusting the atrial-ventricular delay duration of the target pacemaker is greater than an upper limit value of the first preset number range threshold.

Specifically, in practical application, whether there is a hidden AA interval at present will be detected immediately after the AVD is adjusted according to adjustment strategy 1 or adjustment strategy 2, and if there is, the adjustment of AVD will be maintained until the hidden AA interval is not detected within a period or the “confirmation period of atrial tachycardia” ends.

Specifically, the detection of the hidden AA interval is: after adjusting AVD by adjusting strategy 1, if AS/AP-VP-AR sequence is detected immediately and AA interval of this sequence is “atrial tachycardia interval”, it is considered that the hidden AA interval has been detected. In the case that the AVD is adjusted by adjusting strategy 2, if the AS/AP-AR sequence is detected immediately and the AA interval of this sequence is “atrial tachycardia interval”, it is considered that the hidden AA interval has been detected.

Specifically, in an embodiment, after the execution of the above step S104, in the case that the total number of intervals belonging to atrial tachycardia intervals for each historical detection intervals within the set time period after adjusting the atrial-ventricular delay interval duration of the target pacemaker is greater than the upper limit value of the first preset number range threshold, determining that an atrial tachycardia event occurs, it specifically further comprises the following steps:

Step S105: adjusting the target pacemaker from a tracking mode to a non-tracking mode after determining that an atrial tachycardia event occurs.

Specifically, in practical application, after the occurrence of the atrial tachycardia event is determined, the target pacemaker will be adjusted from tracking mode to non-tracking mode until the atrial tachycardia is over.

Specifically, as shown in FIG. 2, the implantable medical device system comprises an implantable medical device (i.e. a target pacemaker) 1 and an in vitro device 10, wherein the target pacemaker 1 consists of a ventricular sensing module 2, a ventricular pacing module 3, a wireless programmed control module 4, a clock/timing module 5, a pacing control module 6, a storage module 7 and a treatment control module 9. Wherein, the ventricular sensing module 2 senses the heart's spontaneous signal and transmits the sensed signal to the pacing control module 6. The clock/timing module 5 records the time when the atrial sensing module 2 senses the heart's spontaneous signal, and the pacing control module 6 calculates the current AA interval according to the time recorded by the clock/timing module 5, and controls the clock/timing module 5 to time the pacing interval according to the current running state at the same time. In the case that the clock/timing module 5 expires, it informs the pacing control module 6, and the pacing control module 6 controls the atrial pacing module 3 to issue atrial pacing pulses according to the current running parameters (such as the current is in tracking mode). The treatment control module 9 can modify operation parameters in the storage module 7 (for example, switch the operation mode to the non-tracking mode after atrial tachycardia is detected) according to the patient status (such as whether atrial tachycardia occurs or not) that the current treatment function (such as atrial tachycardia detection function) runs or evaluates, and the operation parameters in the storage module 7 are updated and transmitted to the pacing control module 6, and the pacing control module 6 controls pacing timing and pacing pulse issue according to the new operation parameters.

The in vitro device 10 is connected with the wireless programmed control module 4 of the target pacemaker 1 by wireless communication, and the in vitro device 10 can modify the operating parameters in the implantable medical device through wireless communication, and the target pacemaker 1 can send internal data to the in vitro device 10 through the wireless programmed control module 4.

By performing the above steps, the method for detecting atrial tachycardia provided by the embodiments of the present application, by monitoring a total number of historical detection intervals belonging to atrial tachycardia intervals within a set time of a target pacemaker in a tracking mode; in the case that the total number is within a first preset number range threshold, adjusting an atrial-ventricular delay interval duration of the target pacemaker; determining whether the total number of intervals belonging to atrial tachycardia intervals for each historical detection intervals within the set time period after adjusting the atrial-ventricular delay interval duration of the target pacemaker is greater than an upper limit value of the first preset number range threshold; in the case that the total number of intervals belonging to atrial tachycardia intervals for each historical detection intervals within the set time period after adjusting the atrial-ventricular delay interval duration of the target pacemaker is greater than the upper limit value of the first preset number range threshold, an atrial tachycardia event to occur is determined. By detecting the total number of intervals belonging to atrial tachycardia intervals for each historical detection intervals within the set time period, in the case that the total number is within the first preset number range threshold, it is suspected that atrial tachycardia event has occurred, and the detection interval hidden in the post ventricular atrial blanking period is detected by adjusting the atrial-ventricular delay duration of the target pacemaker; in the case that the current detection interval is detected as atrial tachycardia interval, continue to determine whether the total number of intervals belonging to atrial tachycardia intervals for each historical detection intervals within the set time period after adjusting the atrial-ventricular delay interval duration of the target pacemaker is greater than an upper limit value of the first preset number range threshold, and in the case that the total number of intervals belonging to atrial tachycardia intervals for each historical detection intervals within the set time period after adjusting the atrial-ventricular delay interval duration of the target pacemaker is greater than the upper limit value of the first preset number range threshold, it can be determined that atrial tachycardia event has occurred. By adjusting the atrial-ventricular delay of the target pacemaker, the hidden atrial tachycardia event can be effectively detected, which improves the detection accuracy of the atrial tachycardia event, thus greatly reducing the occurrence of the patient's discomfort caused by the target pacemaker being at the operating rate for a long time.

The method for detecting atrial tachycardia provided by the embodiments of the present application will be described in detail with specific application examples.

As shown in FIG. 1-3, the specific implementation process is as follows:

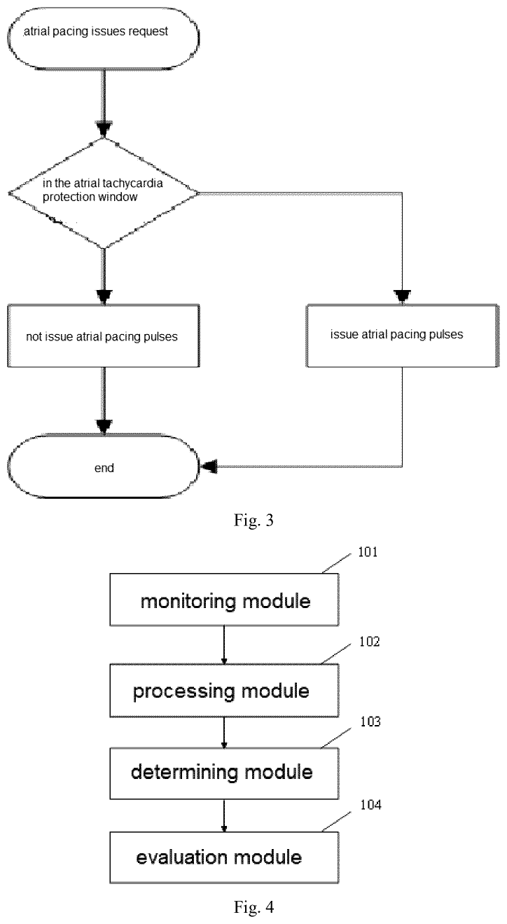

1. In the case that the target pacemaker detects a first atrial rate which exceeds an atrial tachycardia detect rate (ATDR), it opens an atrial tachycardia protection window (ATPW), and opens the window after every subsequent atrial sensing event until N consecutive atrial rates (such as 8) are all lower than the atrial tachycardia detect rate. In this protection window, the atrial pacing pulse issuing is suppressed, and the ventricular pacing rate works according to the basic rate or the sensor indication rate, as shown in FIG. 3. The length of this window is M % (such as 70%) of the period length corresponding to the current atrial tachycardia detect rate.

The specific processing procedure is as follows:
(1) atrial sensing events are detected (detecting atrial sensing events before and after the post ventricular atrial blanking period, the same principle applies below);
(2) determining whether the current AA interval is an atrial tachycardia interval;

If yes, open the “atrial tachycardia protection window”; If not, it is further determined whether N consecutive AA intervals are not atrial tachycardia intervals; if N consecutive AA intervals are not atrial tachycardia intervals, the atrial tachycardia protection window is closed; otherwise, the atrial tachycardia protection window is kept open.

In practical application, the interval of “atrial sensing-atrial pacing” which exceeds the atrial tachycardia detect rate is not considered to reflect the heart rate itself, so it should not be included in the detection and evaluation of atrial tachycardia. This protection window can prevent inappropriate atrial pacing from interfering with the detection of atrial tachycardia events, and at the same time can prevent inappropriate atrial pacing from continuing to maintain or aggravate atrial heartbeat as a new exciting source. Taking the window length as a percentage of the period length corresponding to the current atrial tachycardia detect rate can more reasonably configure actuating range of the window.

2. The atrial tachycardia detect rate can be dynamically adjusted according to the current basic rate or the sensor indication rate (in the case that the sensor indication rate function is turned on), and Xppm is added on the basis of the current basic rate or sensor indication rate, wherein Xppm is a doctor's programmed value and the rate is not lower than the upper limit tracking rate.

During a exercise period, the demand of the cardiac output is relatively large, which requires a higher rate. If the atrial tachycardia detect rate at this time cannot meet the higher rate demand during the exercise period, then improper atrial tachycardia determination may occur during the exercise period, which may lead to improper mode conversion, thus failing to meet the higher rate demand during the exercise period. This technical feature can effectively adapt to tachycardia detection under different operating requirements.

3. If the target pacemaker detects that the following criteria are satisfied at the same time, it is considered that an atrial tachycardia event has been detected.

Detection criterion 1: There are five or more atrial tachycardia intervals in the latest eight AA intervals, wherein the atrial tachycardia interval is defined as:
A1. An AA interval which exceeds the atrial tachycardia detect rate is defined as an atrial tachycardia interval;
A2. If (AA_intvl/2) is less than ATDR_intvl and less than (SAV+PAVB), it is defined as 0.5 atrial tachycardia interval, and this AA interval does not participate in the determination of the following detection criterion 2;

Detection criterion 2: There are three or more AA intervals in the latest eight AA intervals that are atrial tachycardia intervals, and there is no conducted ventricular events.

The specific processing procedure is as follows:
(1) atrial sensing events are detected;
(2) determining whether it is in the confirmation period of atrial tachycardia event:

If it is not in the confirmation period of atrial tachycardia event, the atrial tachycardia event is detected normally, and the detection process is as follows: whether the detection criterion 1 is satisfied or not is determined; if it is satisfied, whether the detection criterion 2 is satisfied or not is determined; if it continues to be satisfied, it is considered that the atrial tachycardia event is detected, and the target pacemaker performs mode conversion to switch the tracking mode to the non-tracking mode. In the case that only detection criterion 1 or detection criterion 2 is satisfied, it is considered to be within a period during which the atrial tachycardia event is suspected, and there may be a hidden AA interval, so it is necessary to detect the hidden AA interval and enter the detection process of suspected atrial tachycardia events. If detection criterion 1 and detection criterion 2 are not satisfied at the same time, it is determined that atrial tachycardia events have not occurred.

Atrial rate exceeding the atrial tachycardia detect rate can not simple be used to accurately determine the occurrence of atrial tachycardia events. Usually, atrial tachycardia events are accompanied by AV Block or 2:1 conduction, so by combining atrial rate and whether atrial events are conducted to make comprehensive determination, atrial tachycardia events can be detected more accurately.

4. If the target pacemaker detects that one of the two criteria in “3” is satisfied and continuous in the “atrial tachycardia suspicion window” (this interval is used to identify whether the suspected phenomenon is continuous, and if it is single, it is ignored), then it is suspected that an atrial tachycardia event has occurred. At this time, it is suspected that atrial sensing events are shielded by the post atrial ventricular blanking period. At this time, by adjusting the atrial-ventricular delay (AVD), it is determined whether there is AA interval hidden by the post atrial ventricular blanking period (PVAB). At the same time, start a confirmation period of atrial tachycardia event, the length of which can be programmed controlled by a doctor, such as 10s. This interval is used to confirm whether the hidden AA interval found after the detection process (determining after adjusting AVD, as described below) is continuous, and only there are continuous hidden AA intervals can it be finally confirmed that the suspicion is established, that is, an atrial tachycardia event occurs. A detection process of the hidden AA interval is as follows:

Adjustment strategy 1: If the current minimum AA interval is greater than SAVD+(PVAB/2) and less than SAVD+PVAB, decrease AVD=AVD−(PVAB/2), wherein the minimum AVD is not less than 30 ms; whether there is a hidden AA interval at present will be detected immediately after the AVD is adjusted, and if there is, the adjustment of AVD will be maintained until the hidden AA interval is not detected within a period or the confirmation period of atrial tachycardia event ends. Wherein, if AS/AP-VP-AR sequence is detected and the current AA interval is “atrial tachycardia interval”, it is considered that the hidden AA interval has been detected.

Adjustment strategy 2: If the current minimum AA interval is greater than SAVD and less than SAVD+½PVAB, expand AVD=AVD+(PVAB/2), and the maximum AVD is not more than 300 ms; whether there is a hidden AA interval at present will be detected immediately after the AVD is adjusted, and if there is, the adjustment of AVD will be maintained until the hidden AA interval is not detected within a period or the confirmation period of atrial tachycardia event ends. Wherein, if AS/AP-AR sequence is detected and the current AA interval is “atrial tachycardia interval”, it is considered that the hidden AA interval has been detected.

Wherein, the AVD includes SAVD and PAVD. After adjustment by the above adjustment strategy 1 or 2, it is immediately confirmed that the atrial tachycardia event is detected if they satisfied the detection criteria described in “3” at the end of the confirmation period of atrial tachycardia event. Otherwise, directly exit the confirmation period of atrial tachycardia event.

Wherein, AVD+½PVAB interval is used to distinguish whether atrial sensing is shielded in the first half or the second half of PVAB. If it is in the first half, AVD is expanded, and if it is in the second half, AVD is decreased, so that whether there is a hidden AA interval can be detected more specifically. Detecting whether there is a hidden AA interval immediately after each adjustment can prevent abnormal cardiac timing caused by long-term ineffective AVD expansion.

5. After the process described in “4” above, the confirmation period of atrial tachycardia event and the detection process of hidden AA interval are started. If the confirmation period of atrial tachycardia event is detected at this time, it is further detected whether the confirmation period of atrial tachycardia event has expired (the length of the confirmation period of atrial tachycardia is programmed controlled by the doctor, for example, 10s):

(1) If it has not expired, it is still in the process of confirmation of suspicion. At this time, it is further determined whether a detection process of hidden AA interval is started:

\textcircled{1} If it is started, the hidden AA interval is determined (the determining process is as described in “4” above); if it is determined that there is a hidden AA interval, the detection process of hidden AA interval is maintained; if it is determined that there is no hidden AA interval, the detection process of hidden AA interval is closed and AVD is resumed. At this time, it is determined that there is no hidden AA interval, so it is not necessary to continue to maintain the adjustment of AVD, and there is no need to perform the detection process of hidden AA interval, which can prevent abnormal cardiac timing caused by long-term ineffective AVD expansion.

\textcircled{2} If it is not started (the detection process of hidden AA interval is closed in the process of \textcircled{1}, it will directly exit and wait for the confirmation period of atrial tachycardia event to expire.

(2) if it expires, it is further determined whether the detection criterion 1 and the detection criterion 2 are satisfied after the detection process of hidden AA interval, and if it is satisfied, it is considered that an atrial tachycardia event has been detected, and mode conversion is performed; Otherwise, if it is not satisfied, just exit the confirmation period of atrial tachycardia events directly, and then continue to detect atrial tachycardia events according to the process.

Once the atrial tachycardia event is detected, the mode is immediately switched to the non-tracking mode until the atrial tachycardia is over.

By monitoring a total number of historical detection intervals belonging to atrial tachycardia intervals within a set time of a target pacemaker in a tracking mode; in the case that the total number is within a first preset number range threshold, adjusting an atrial-ventricular delay interval duration of the target pacemaker; determining whether the total number of intervals belonging to atrial tachycardia intervals for each historical detection intervals within the set time period after adjusting the atrial-ventricular delay interval duration of the target pacemaker is greater than an upper limit value of the first preset number range threshold; in the case that the total number of intervals belonging to atrial tachycardia intervals for each historical detection intervals within the set time period after adjusting the atrial-ventricular delay interval duration of the target pacemaker is greater than the upper limit value of the first preset number range threshold, an atrial tachycardia event to occur is determined. The detection interval in the post ventricular atrial blanking period is detected by adjusting the atrial-ventricular delay duration of the target pacemaker; in the case that the total number of intervals belonging to atrial tachycardia intervals for each historical detection intervals within the set time period is greater than the upper limit value of the first preset number range threshold, it can be determined that atrial tachycardia event has occurred. By combining the two detection criteria, the problem that it can not accurately evaluate atrial tachycardia events by judging atrial rate alone is resolved. By adjusting the duration length of atrial-ventricular delay in time and detecting the hidden AA interval, the problem that atrial sensing events shielded by PVAB cannot be identified is solved. By introducing the atrial tachycardia protection window, the problem that atrial pacing events issued by pacemakers may lead to misjudgment of “atrial tachycardia events” in the case that atrial tachycardia is suspected is solved. While effectively identifying shielded atrial sensing events, the detection accuracy of atrial tachycardia events is improved, and the occurrence of the patient's discomfort caused by the target pacemaker being at the high operating rate for a long time is greatly reduced.

The embodiments of the present application provide an apparatus for detecting atrial tachycardia, as shown in FIG. 4, which comprise:

A detection module 101, configured to monitoring a total number of intervals belonging to atrial tachycardia intervals for each historical detection intervals within a set time period of a target pacemaker in a tracking mode. For details, please refer to the related description of step S101 in the above-mentioned method embodiment, which will not be repeated here.

A processing module 102, configured to adjust an atrial-ventricular delay duration of the target pacemaker in the case that the total number is within a first preset number range threshold. For details, please refer to the related description of step S102 in the above-mentioned method embodiment, which will not be repeated here.

A determining module 103, configured to determine whether the total number of intervals belonging to atrial tachycardia intervals for each historical detection intervals within the set time period after adjusting the atrial-ventricular delay duration of the target pacemaker is greater than an upper limit value of the first preset number range threshold. For details, please refer to the related description of step S103 in the above-mentioned method embodiment, which will not be repeated here.

An evaluation module 104, configured to determine that an atrial tachycardia event occurs in the case that the total number of intervals belonging to atrial tachycardia intervals for each historical detection intervals within the set time period after adjusting the atrial-ventricular delay duration of the target pacemaker is greater than the upper limit value of the first preset number range threshold. For details, please refer to the related description of step S104 in the above-mentioned method embodiment, which will not be repeated here.

For a further description of the above-mentioned apparatus for detecting atrial tachycardia, please refer to the relevant description of the above-mentioned method embodiments for detecting atrial tachycardia, which will not be repeated here.

Through the cooperation of the above components, the apparatus for detecting atrial tachycardia provided by the embodiments of the present application, by detecting the total number of intervals belonging to atrial tachycardia intervals for each historical detection intervals within the set time period, in the case that the total number is within the first preset number range threshold, it is suspected that atrial tachycardia event has occurred, and the detection interval hidden in the post ventricular atrial blanking period is detected by adjusting the atrial-ventricular delay duration of the target pacemaker; in the case that the total number of intervals belonging to atrial tachycardia intervals for each historical detection intervals within the set time period after adjusting the atrial-ventricular delay interval duration of the target pacemaker is greater than the upper limit value of the first preset number range threshold, it can be determined that atrial tachycardia event has occurred. By adjusting the atrial-ventricular delay of the target pacemaker, the hidden atrial tachycardia event can be effectively detected, which not only improves the detection accuracy of the atrial tachycardia event, but also greatly reduces the occurrence of the patient's discomfort caused by the target pacemaker being at the operating rate for a long time.

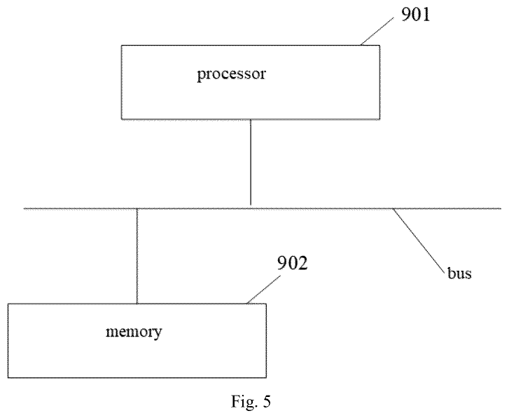

The embodiment of the present application provides an electronic device, as shown in FIG. 5, which comprises a processor 901 and a memory 902, and the memory 902 and the processor 901 are in communication connection with each other, wherein the processor 901 and the memory 902 can be connected by a bus or other means, and the connection by a bus is taken as an example in FIG. 5.

The processor 901 may be a Central Processing Unit (CPU). The processor 901 can also be other general-purpose processors, Digital Signal Processor (DSP), application specific integrated circuits (ASIC), Field-Programmable Gate Array (FPGA) or other programmable logic devices, discrete gate or transistor logic devices, discrete hardware components and other chips, or a combination of the above-mentioned chips.

As a non-transient computer-readable storage medium, the memory 902 can be used to store non-transient software programs, non-transient computer-executable programs and modules, such as program instructions/modules corresponding to the method in the embodiment of the present application. The processor 901 executes various functional applications and data processing of the processor 901 by running non-transient software programs, instructions and modules stored in the memory 902, that is, the method in the above method embodiment is implemented.

The memory 902 may comprise a storage program area and a storage data area, wherein the storage program area may store an operating system and an application program required by at least one function; The storage data area may store data created by the processor 901 and the like. In addition, the memory 902 may comprise high-speed random access memory and non-transient memory, such as at least one disk memory device, flash memory device, or other non-transient solid-state memory devices. In some embodiments, the memory 902 may optionally comprise memories located remotely from the processor 901, and these remote memories may be connected to the processor 901 through a network. Examples of the above networks include, but are not limited to, the Internet, an intranet, a local area network, a mobile communication network, and combinations thereof.

One or more modules are stored in the memory 902, and in the case that executed by the processor 901, the method in the above method embodiment is executed.

The specific details of the above-mentioned electronic device can be understood by referring to the corresponding related descriptions and effects in the above-mentioned method embodiments, and will not be repeated here.

It can be understood by persons skilled in the art that all or part of the processes in the methods of the above-mentioned embodiments can be completed by instructing related hardware through a computer program, and the implemented program can be stored in a computer-readable storage medium, which, in the case that executed, can comprise the processes of the above-mentioned methods. The storage medium can be a magnetic disk, an optical disk, a Read-Only Memory (ROM), a Random Access Memory (RAM), a Flash Memory, a Hard Disk Drive (HDD) or a Solid-State Drive (SSD). The storage medium may also comprise a combination of the above kinds of memories.

Apparently, the above-mentioned embodiments are only examples for clear explanation, and are not a limitation of the implementation. For persons skilled in the art, other changes or changes in different forms can be made on the basis of the above description. It is not necessary and impossible to exhaust all the embodiments here. However, the obvious changes or changes caused by this are still within the protection scope of the present application.

1. A method for detecting atrial tachycardia, comprising:
monitoring a total number of historical detection intervals belonging to atrial tachycardia intervals within a set time of a target pacemaker in a tracking mode;
in the case that the total number is within a first preset number range threshold, adjusting an atrial-ventricular delay interval duration of the target pacemaker;
determining whether the total number of historical detection intervals belonging to the atrial tachycardia intervals within the set time after the atrial-ventricular delay interval duration of the target pacemaker is adjusted is greater than an upper limit value of the first preset number range threshold; and
in the case that the total number of the historical detection intervals belonging to the atrial tachycardia intervals within the set time after the atrial-ventricular delay interval duration of the target pacemaker is adjusted is greater than the upper limit value of the first preset number range threshold, determining that an atrial tachycardia event occurs.

2. The method according to claim 1, further comprising before the step of determining whether the total number of historical detection intervals belonging to the atrial tachycardia intervals within the set time after the atrial-ventricular delay interval duration of the target pacemaker is adjusted is greater than an upper limit value of the first preset number range threshold:
starting a confirmation period of atrial tachycardia;
acquiring a detection sequence of a current detection interval, and determining whether a sequence of the detection sequence is consistent with that of a target sequence;
in the case that the sequence of the detection sequence is consistent with that of the target sequence, maintaining the adjustment of the atrial-ventricular delay interval duration of the target pacemaker;
determining whether the current detection interval is the atrial tachycardia interval; and
in the case that the current detection interval is the atrial tachycardia interval and the current detection interval is within the confirmation period of atrial tachycardia, determining whether the total number of historical detection intervals belonging to the atrial tachycardia within the set time after the atrial-ventricular delay interval duration of the target pacemaker is adjusted is greater than an upper limit value of the first preset number range threshold.

3. The method according to claim 1, wherein the step of monitoring a total number of historical detection intervals belonging to atrial tachycardia intervals within a set time of a target pacemaker in a tracking mode comprises:
acquiring an atrial sensing event in the current historical detection interval within a set time;
determining whether a rate of the atrial sensing event exceeds a atrial tachycardia detect rate;
determining that the current historical detection interval is an atrial tachycardia interval in the case that the rate of the atrial sensing event exceeds the atrial tachycardia detect rate; and
counting a total number of all atrial tachycardia intervals.

4. The method according to claim 3, wherein before the step of counting a total number of all atrial tachycardia intervals, the method further comprises:
in the case that the rate of the atrial sensing event does not exceed the atrial tachycardia detect rate, determining whether the rate of the atrial sensing event is greater than a second detect rate, wherein the second detect rate is less than the atrial tachycardia detect rate; and
in the case that rate of the atrial sensing event is greater than the second detect rate, determining that the current historical detection interval corresponding to the atrial sensing event is a preset number of atrial tachycardia intervals, wherein the preset number is greater than 0 and less than 1.

5. The method according to claim 1, wherein the step of adjusting an atrial-ventricular delay interval duration of the target pacemaker comprises:
obtaining durations of a post ventricular atrial blank period and an atrioventricular interval; and
adjusting the atrial-ventricular delay interval duration based on a relationship between the current detection interval and the durations of the post ventricular atrial blank period and the atrioventricular interval.

6. The method according to claim 4, wherein in the case that the total number is greater than the upper limit value of the first preset number range threshold, the method further comprises:
determining whether a number of atrial tachycardia intervals identified as integer in the total number is greater than a second preset number threshold;
in the case that the number is greater than the second preset number threshold, determining whether a ventricular event occurs in each historical detection interval in which the value of the atrial tachycardia interval is identified as an integer within the set time of the monitoring target pacemaker in the tracking mode; and
in the case that the number of ventricular events not occurring in the historical detection intervals is greater than the second preset number threshold, determining that an atrial tachycardia event occurs.

7. The method according to claim 1, wherein the method further comprises:
adjusting the target pacemaker from a tracking mode to a non-tracking mode after determining that an atrial tachycardia event occurs.

8. (canceled)

9. An electronic device, comprising:
a memory and a processor, wherein the memory and the processor are in communication connection with each other, and computer instructions are stored in the memory, and the processor executes the method according to claim 1 by executing the computer instructions.

10. A non-transitory computer-readable storage medium, wherein the computer-readable storage medium stores computer instructions for causing the computer to execute the method according to claim 1.

11. A non-transitory computer-readable storage medium, wherein the computer-readable storage medium stores computer instructions for causing the computer to execute the method according to claim 2.

12. A non-transitory computer-readable storage medium, wherein the computer-readable storage medium stores computer instructions for causing the computer to execute the method according to claim 3.

13. A non-transitory computer-readable storage medium, wherein the computer-readable storage medium stores computer instructions for causing the computer to execute the method according to claim 4.

14. A non-transitory computer-readable storage medium, wherein the computer-readable storage medium stores computer instructions for causing the computer to execute the method according to claim 5.

15. A non-transitory computer-readable storage medium, wherein the computer-readable storage medium stores computer instructions for causing the computer to execute the method according to claim 6.

16. A non-transitory computer-readable storage medium, wherein the computer-readable storage medium stores computer instructions for causing the computer to execute the method according to claim 7.
